(19) United States
(12) Patent Application Publication
(10) Pub. No.: US 2024/0310366 A1
(43) Pub. Date: Sep. 19, 2024

(54) Mirror Aptamer-Based Biosensors

(71) Applicant: BioBoost Synbio Consulting Inc., Burnaby (CA)

(72) Inventors: Nawwaf Kharma, Burnaby (CA); Gabriel Aguiar-Tawil, Burnaby (CA)

(21) Appl. No.: 18/543,083

(22) Filed: Dec. 18, 2023

Related U.S. Application Data
(60) Provisional application No. 63/434,263, filed on Dec. 21, 2022.

(51) Int. Cl. G01N 33/53; C12N 15/113; G01N 33/542

(52) U.S. Cl. CPC G01N 33/5308; C12N 15/113; G01N 33/542; C12N 2310/16

(57) ABSTRACT

A nucleic acid molecule, for example, DNA, aptamer-based molecular sensing system is made up of three components: the aptamer, the trigger, and the sensing strand.

### PRIOR APPLICATION INFORMATION

The instant application claims the benefit of US Provisional Patent Application U.S. Ser. No. 63/434,263, filed Dec. 21, 2022 and entitled “MIRROR APTAMER-BASED BIOSENSORS”, now abandoned, the entire contents of which are hereby incorporated herein by reference for all purposes.

### BACKGROUND OF THE INVENTION

Aptamers are short single stranded DNA or RNA molecules that can bind to a broad range of compounds of interest, or targets, such as small molecules or proteins, with high affinity and selectivity.

Aptamers for a large number of compounds of interest are well-known in the art, as are methods for the generation of same as well as their use in biosensors, where different reporters are used.

For example, Wang et al (2019, Chinese Chemical Letters 30: 1017-1020) teaches an aptamer comprising a molecular beacon. Specifically, there is no target induced displacement and the molecular beacon is active but upon target binding, the fluorophore and quencher come together, quenching the signal. Furthermore, this method does not allow for amplification, therefore, the detection limit of this technology is limited to the sensitivity of the aptamer.

Pei (2011, 2010 International Conference on Nanotechnology and Biosensors 2: 143-147) uses target induced displacement with a molecular beacon-type system wherein the molecular beacon is separated such that the complement strand has the quencher and the aptamer has a fluorophore so that target binding releases complement and allows the aptamer to glow. However, this method does not allow for amplification, as one Q-tipped complement is released for every molecule binding to the F-tipped aptamer. Furthermore, this system requires new oligos (for both the F-tipped aptamer and Q-tipped complement) for every new molecule.

Jalalian et al (2021, Spectrochimica Acta Part A: Molecular and Biomolecular Spectroscopy 246: 119062) teaches a biosensor wherein the complement strand is bound to the aptamer. Upon target binding, the complement strand is released and stabilizes gold nanoparticles, preventing colour change. However, this method has a centrifugation step, meaning that lab equipment is required for the method, thereby limiting its usefulness in point-of-care applications

Prante et al (2019, Biosensors 9:134) teaches a biosensor that uses target induced displacement for the detection of a target molecule. However, this method requires new oligos to be constructed for each molecule and does not allow for amplification. Furthermore, the method is based on a negative indication, that is, a drop in fluorescence, for detection.

Munzar et al (2018, Nature Communications 9:343) discusses the thermodynamics of “triggers” which can be used to prepare design rules.

Zheng et al (2018, Chem Res Chin Univ 34: 952-958) teaches a biosensor that uses target induced displacement. Specifically, a complementary strand of DNA is bound to the aptamer initially, but, upon target binding, the complement is released. A molecular beacon then binds to the complement, releasing a signal. However, this method appears to require some trial and error and tuning is needed for different target molecules.

Nutiu and Li (2005, Angew Chem Int Ed 44: 1061-1065) teaches a biosensor that uses target induced displacement with an extended aptamer sequence. However, this method requires the use of a functional SELEX for each new molecule and offers no possibility of amplification.

Yang et al (2014, Nature Chemistry 6: 1003-1008) teaches a biosensor that uses target induced displacement to differentiate different aptamers for monosaccharides. This method also provides no possibility of amplification

Han et al (2008, Electrochemistry Communications 11: 157-160) teaches a biosensor wherein the complement strand is attached to a solid support and bound to aptamer. Upon binding to a target, the aptamer is removed, and the complement is left free. A signal strand of DNA modified with a redox-reactive molecule then binds to the complement. This allows the redox-reactive molecule to interact with the electrode and produce a signal. In this method, the oligo that is released upon binding of the molecule is the apatamer, which is not amplifiable. The complement that stays is linked to a solid surface, and is assessed electrically for binding or not binding to a ferrocene-tipped DNA strand.

Esmaelpourfarkhani et al (2020, Biosensors and Bioelectronics 164: 112329) teaches a biosensor that uses target induced displacement from a gold nanoparticle solid support. Specifically, the aptamer is bound to the gold nanoparticle, but the ligand or target molecule removes the complement to the aptamer. The displaced complement quenches dissolved fluorophore. This method also requires a centrifugation step, meaning that a centrifuge is required, limiting usefulness of this method in point-of-care applications.

### SUMMARY OF THE INVENTION

According to an aspect of the invention, there is provided a biosensor for detecting a compound of interest, said biosensor comprising:
an aptamer, said aptamer comprising a target binding region configured to be bound by or to bind to a compound of interest;
a sensor, said sensor comprising a sensing sequence region and a detectable reporter, said sensor having a reporting configuration wherein the reporter is detected and a non-reporting configuration; and
a trigger, said trigger comprising an aptamer binding region that binds to the target binding region and at least one sensing sequence binding region that binds to the sensing sequence, said trigger having an aptamer binding configuration wherein the aptamer binding region is bound to the target binding region of the aptamer and a sensor binding configuration wherein the at least one sensing sequence binding region is bound to the sensing sequence region of the sensor.

According to another aspect of the invention, there is provided a method for detecting a compound of interest with a biosensor comprising:
providing a biosensor comprising:
   an aptamer, said aptamer comprising a target binding region configured to be bound by or to bind to a compound of interest;
   a sensor, said sensor comprising a sensing sequence region and a detectable reporter, said sensor having a reporting configuration wherein the reporter is detected and a non-reporting configuration; and
   a trigger, said trigger comprising an aptamer binding region that binds to the target binding region and at least one sensing sequence binding region that binds to the sensing sequence, said trigger having an aptamer binding configuration wherein the aptamer binding region is bound to the target binding region of the aptamer and a sensor binding configuration wherein the at least one sensing sequence binding region is bound to the sensing sequence region of the sensor;
to a sample suspected of comprising the compound of interest, adding the biosensor wherein the biosensor is configured such that the sensor is in the non-reporting configuration and the trigger is in the aptamer binding configuration;
incubating the sample comprising the biosensor under conditions such that the compound of interest, if present, binds to the target binding region of the aptamer of a respective one biosensor,
said binding of the compound of interest to the target binding region of the aptamer of the respective one biosensor releasing the aptamer binding region of the trigger from binding to the target binding region of the aptamer;
said released trigger binding to the sensing sequence region of the sensor such that the at least one sensing sequence binding region of the trigger interacts with and binds to the sensing sequence region of the sensor,
said binding of the released trigger to the sensing sequence region converting the trigger to the sensor binding configuration and converting the sensor to the reporting configuration; and
detecting the reporter.

### BRIEF DESCRIPTION OF THE DRAWINGS

FIG. 1: Schematic diagram of the biosensor in the inactive state with the aptamer bound to the trigger with wings extending from each end of the bound regions of the trigger.

FIG. 2: Schematic diagram showing a target molecule binding to the aptamer, causing release of the trigger molecule.

FIG. 3: Schematic diagram showing a sensor with a molecular beacon in a non-reporting configuration and a trigger molecule. Binding of the trigger molecule to the sensor with the molecular beacon changes the secondary structure of the sensor, 5 making the molecular beacon active.

FIG. 4: Schematic diagram showing an embodiment of the invention. (A) The trigger has a single wing and is bound to the DNA aptamer; (B) the biosensor system includes additional components, specifically a fluorescent aptamer template, in this case, a template for transcription by the T7 promoter, and a short partial primer corresponding to a first region of the T7 promoter; (C) the trigger is dislodged or dislocated or released from the aptamer; (D) the released aptamer binds to its target; (E) the RNA transcript produced from the fluorescent aptamer template is an RNA molecule or strand that binds a fluorescent substrate.

FIG. 5. Schematic diagram showing the biosensor system with a single wing trigger and an alternative reporter (gold nanoparticles). (A) shows the biosensor comprising an aptamer and a trigger as shown in FIG. 1. (B) Reporter comprising gold nanoparticles bound by a shield DNA. (C) Displacement of the trigger from the aptamer by the target. (D) Sensing sequence binding region of the trigger binds to the shield DNA, forming a duplex and releasing the gold nanoparticle. (E) Detection of gold nanoparticles.

FIG. 6: Bar graph showing response of ampicillin system when treated with 50 μM ampicillin.

FIG. 7: Graph of response of kanamycin system when treated with kanamycin.

FIG. 8: Bar graph showing comparison of fluorescence response of kanamycin and related compounds.

FIG. 9: Graph of fluorescent response of cocaine system due to treatment with cocaine.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, “annealing” refers to the interaction between two single stranded nucleic acid sequences which form a double stranded sequence.

As used herein, “target” refers to a compound or molecule of interest.

As used herein, “aptamer” refers to a single stranded DNA or RNA oligonucleotide that binds to a target molecule.

As used herein, “trigger” refers to a piece of DNA annealed to the aptamer that releases upon the target binding to the aptamer.

As used herein, “stem” refers to a double stranded secondary structure of an oligonucleotide molecule caused by it annealing to itself.

As used herein, “sensing strand” refers to a nucleic acid molecule or portion thereof that produces a signal or a change in a signal when bound to a trigger.

As used herein, a “molecular beacon probe” refers to an oligonucleotide probe in which one end comprises a fluorophore and the other end comprises a quencher. Upon annealing to a complementary sequence, the quencher and probe are separated, allowing for fluorescence.

As used herein, “sensitivity” refers to the ability to detect a range of concentrations.

As used herein, “specificity” refers to the ability to target only the intended molecule.

Described herein is a novel method of nucleic acid molecule, for example, DNA, aptamer-based molecular sensing. This system is made up of three components: the aptamer, the trigger, and the sensing strand.

According to an aspect of the invention, there is provided a biosensor for detecting a compound of interest, said biosensor comprising:
an aptamer, said aptamer comprising a target binding region configured to be bound by or to bind to a compound of interest;
a sensor, said sensor comprising a sensing sequence region and a detectable reporter, said sensor having a reporting configuration wherein the reporter is detected and a non-reporting configuration; and
a trigger, said trigger comprising an aptamer binding region that binds to the target binding region and at least one sensing sequence binding region that binds to the sensing sequence, said trigger having an aptamer binding configuration wherein the aptamer binding region is bound to the target binding region of the aptamer and a sensor binding configuration wherein the at least one sensing sequence binding region is bound to the sensing sequence region of the sensor.

According to another aspect of the invention, there is provided a method for detecting a compound of interest with a biosensor comprising:
providing a biosensor comprising:
   an aptamer, said aptamer comprising a target binding region configured to be bound by or to bind to a compound of interest;
   a sensor, said sensor comprising a sensing sequence region and a detectable reporter, said sensor having a reporting configuration wherein the reporter is detected and a non-reporting configuration; and
   a trigger, said trigger comprising an aptamer binding region that binds to the target binding region and at least one sensing sequence binding region that binds to the sensing sequence, said trigger having an aptamer binding configuration wherein the aptamer binding region is bound to the target binding region of the aptamer and a sensor binding configuration wherein the at least one sensing sequence binding region is bound to the sensing sequence region of the sensor;
to a sample suspected of comprising the compound of interest, adding the biosensor wherein the biosensor is configured such that the sensor is in the non-reporting configuration and the trigger is in the aptamer binding configuration;
incubating the sample comprising the biosensor under conditions such that the compound of interest, if present, binds to the target binding region of the aptamer of a respective one biosensor,
said binding of the compound of interest to the target binding region of the aptamer of the respective one biosensor releasing the aptamer binding region of the trigger from binding to the target binding region of the aptamer;
said released trigger binding to the sensing sequence region of the sensor such that the at least one sensing sequence binding region of the trigger interacts with and binds to the sensing sequence region of the sensor,
said binding of the released trigger to the sensing sequence region converting the trigger to the sensor binding configuration and converting the sensor to the reporting configuration; and
detecting the reporter.

As will be appreciated by those of skill in the art, methods for the generation of aptamers are known in the art. As such, details on methods for the generation of such aptamers is not provided herein, but are within the scope of the invention. That is, any aptamer can be used within the scope of the invention. Furthermore, because the biosensor system of the invention is capable of enhancing the signal by amplification of the trigger sequence, the system provides high degrees of sensitivity and specificity.

That is, as discussed herein, it is important to note that the biosensor system of the invention comprises an intermediate, specifically, the trigger, which greatly improves the flexibility and sensitivity of the system. For example, a variety of different reporter systems can be used because of the intermediary trigger and, as noted above and as discussed in greater detail herein, the intermediary trigger can be amplified in some embodiments, thereby significantly increasing the sensitivity of the biosensor system.

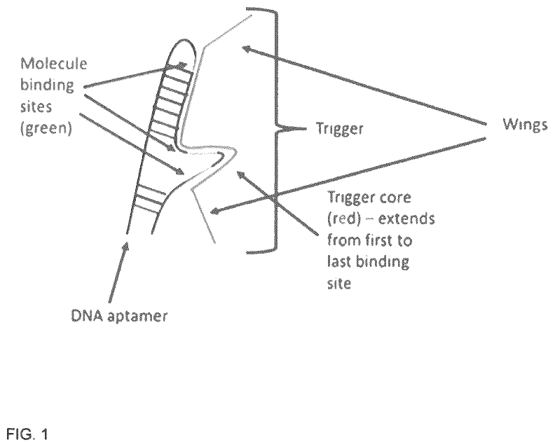

The construction and use of the biosensor system in one embodiment is shown in FIGS. 1-3.

As can be seen, FIG. 1 shows an aptamer and the corresponding trigger, wherein the trigger is in the aptamer binding configuration or conformation, as discussed above.

Specifically, a suitable trigger allows for strong binding of the aptamer binding sequence to the aptamer when the system is in an inactive state, that is, in the aptamer binding configuration, but will release from or be released by the aptamer upon competition for binding with the aptamer by the target. In addition, the at least one sensing sequence binding region must bond or anneal to the sensing sequence region of the sensor upon the release of the trigger from the aptamer. That is, it must be thermodynamically favorable for the trigger to assume the sensor binding configuration upon release or competitive displacement from the aptamer.

As shown in FIG. 1, the aptamer has at least one molecule binding site or target binding site. The aptamer binding region of the trigger is bound to or annealed to the target binding site of the aptamer because that configuration is the most thermodynamically favorable configuration for the trigger to assume when the target binding site of the aptamer is otherwise unoccupied.

In some embodiments of the invention, the trigger is a single strand of DNA or RNA that is:
(1) Perfectly complementary to a continuous region on the aptamer that starts at or before the first base of the first binding site of the target molecule on the 5′-end of the aptamer,
(2) Continues to partially, at least partially or completely complement the sequence of the aptamer corresponding to the target binding region that contains the remaining binding site(s) of the target molecule, if any, and
(3) Ends at or just beyond the last base of the last binding site.

In the embodiment shown in FIG. 1, there are two sensing strand sequence binding regions, which appear in the figure as wings that extend from the aptamer binding region. As such, in this embodiment, the aptamer binding region of the trigger may be referred to as the “core” and the two sensing strand sequence binding regions, each extending from one end of the “core”, may be referred to as “wings”.

As discussed herein, the core or aptamer binding region is extended at either one of or both of its 5′ and 3′ ends by the at least one sensing sequence binding region, said at least one sensing sequence region comprising 5-75 nucleotides.

In embodiments wherein there are two sensing sequence regions, these sensing sequence regions or wings:
(1) May or may not be of equal length (i.e., no. of nucleotides or nt);
(2) May or may not be fully complementary or partially complementary to certain regions of the aptamer; and
(3) Must be at least partially complementary to a nucleotide sequence within the sensing sequence region of the sensor.

As will be appreciated by those of skill in the art, in some embodiments, the sequences of the wing(s) are selected such that the trigger has a co-folded secondary structure that:
(1) Eliminates or at least minimizes base-pairing between the wing(s) and the aptamer; and
(2) Maintains all of the sequence of the trigger-core.

As will be appreciated by one of skill in the art, FIG. 1 is thus a schematic diagram of the aptamer and trigger of the biosensor, wherein the trigger is in the aptamer binding configuration or conformation, that is, wherein the aptamer binding region of the trigger is bound to or annealed to the target binding region of the aptamer and the at least one sensing sequence binding region of the sensor comprises two wings, each extending from one end of the aptamer binding region, and both wings are free or unoccupied.

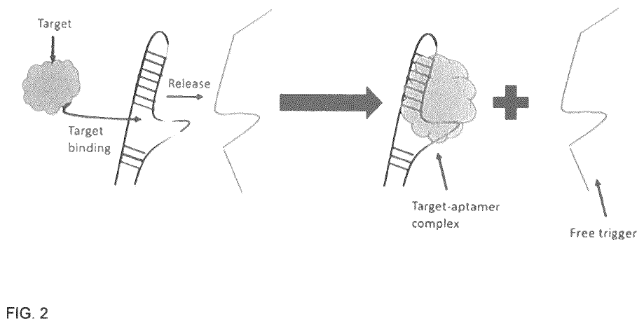

Shown in FIG. 2 is schematic diagram showing what happens to the aptamer and trigger when this part of the biosensor is added to a sample which contains the aptamer's target molecule.

Specifically, as can be seen in FIG. 2, the target molecule and the aptamer binding region of the trigger compete for intermolecular interactions at the target binding domain of the aptamer.

As discussed above, while the aptamer binding domain or core of the trigger is selected for strong binding to the aptamer, on exposure to the aptamer's target molecule, release of the trigger's aptamer binding region by the aptamer so that the aptamer's trigger binding region can bind to the trigger molecule is thermodynamically more favorable.

That is, as shown in the second panel of FIG. 2, the target molecule binds to the aptamer's target binding region and dislodges or displaces the trigger's aptamer binding region from the aptamer. This trigger-mediated displacement of the trigger results in the formation of a target molecule-aptamer complex and a free trigger, as shown in the second panel of FIG. 2.

This sensing strand can either have intrinsic sensing functionality, such as a molecular beacon probe, in which it itself produces a signal or a change in a signal. Alternatively, it can have extrinsic sensing functionality in which the trigger sequence induces a change in another molecule, such as a gold nanoparticle, thereby producing a signal.

Other reporter combinations include but are by no means limited to electrochemical detection, or colourimetric detection via coupling with horseradish peroxidase.

Alternatively, gold nanoparticles can be used as a method of colourimetric sensing. Gold nanoparticles, when not aggregated, are red, and when aggregated, are blue. Single stranded DNA can adsorb onto the surface of gold nanoparticles, protecting them from aggregation while double stranded DNA cannot due to repulsion between their backbone and the gold nanoparticle. So, for example, when the sensing strand is adsorbed onto the surface of the gold nanoparticles, this will protect or prevent the gold nanoparticles from aggregation. However, on release or displacement by the target molecule, the freed trigger binds to the sensing strand, releasing it from the gold nanoparticles, making the gold nanoparticles susceptible to aggregation which in turn will result in a colour change, as discussed below.

As will be appreciated by one of skill in the art, other reporters may be used and are within the scope of the invention.

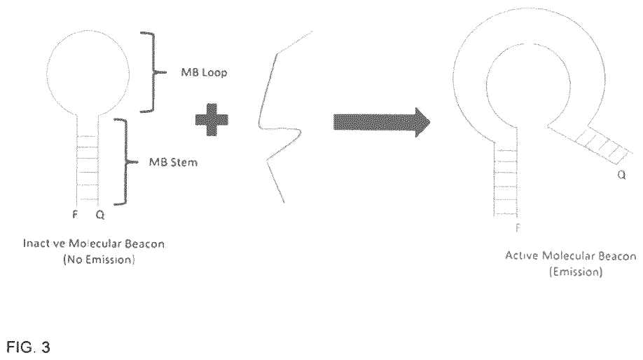

FIG. 3 is a schematic diagram of one embodiment of the invention wherein the sensor is in the non-reporting configuration or conformation with the free trigger initially which then proceeds to a state wherein the trigger is in its sensor binding configuration and the sensor is in the reporting configuration.

In the embodiment shown in FIG. 3, the sensor comprises a molecular beacon as the detectable reporter. Specifically, as can be seen in FIG. 3, in this embodiment, the sensor is a single-stranded nucleic acid that has a fluorophore molecule at a first end thereof and a quenching molecule at the second end thereof.

Furthermore, in this embodiment, the sensor has complementary regions at the 5′ and 3′ ends thereof that form a stem structure separated by a loop (non-binding) sequence. That is, in the embodiment shown in FIG. 3, the sensing sequence region is proximal to the 5′ end and the 3′ end of the sensor and are separated by the loop while the reporter is at either the 3′ end or the 5′ end of the sensor. Furthermore, in the non-reporting configuration, the sensor is configured such that the quencher and the detectable reporter are proximal to each other, so that the detectable reporter is effectively silenced by the quencher or quenching molecule.

In this embodiment, where the sensor comprises a molecular beacon, the sensor is designed such that:
(1) the sequence at the 5′ end of the sensor is complementary to the sequence of the 3′ wing of the trigger;
(2) the loop is a short (5-15 nt) single strand of DNA, preferably free of any internal base-pairing (e.g., CACACA);
(3) the sequence of the 3′ end of the sensor is complementary to the 5′ wing of the trigger.

In some embodiments, the sensing capabilities of this system can be further improved through the amplification of the trigger sequence prior to interacting with the sensing strand. Amplification can be used to decrease the detection limit and increase the dynamic range of the detection system, allowing for increased sensitivity. Amplification is dependent on the trigger strand's ability to transduce the signal. This transduction step allows for either the trigger to be amplified or have the trigger act as a scaffold for degradation of the molecular beacon in some methods. For example, digestion of the sensing strand with a restriction enzyme frees the fluorophore and allows the trigger to be reused (only for molecular beacons). Alternatively, the free trigger could be amplified directly, for example, by transcription-based amplification such as for example by a T7 promoter, as discussed below.

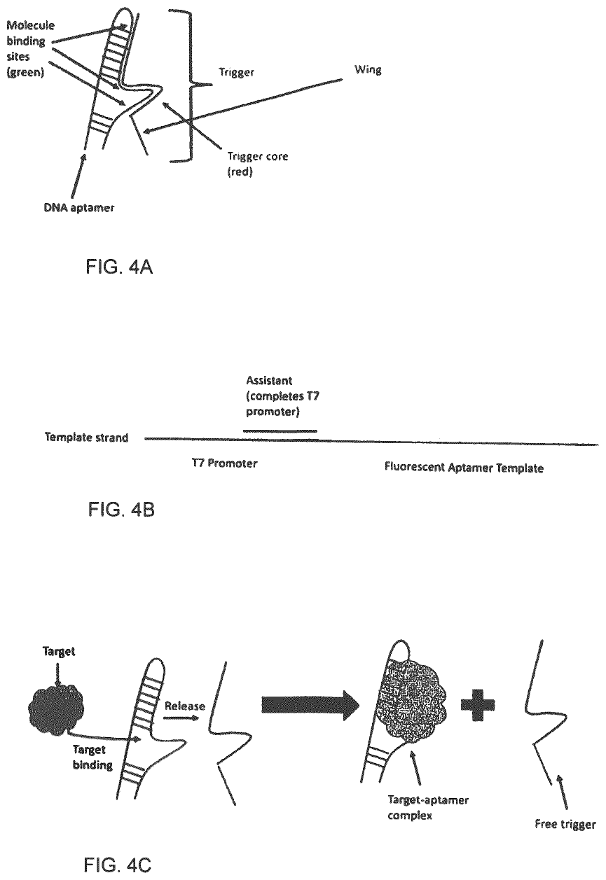

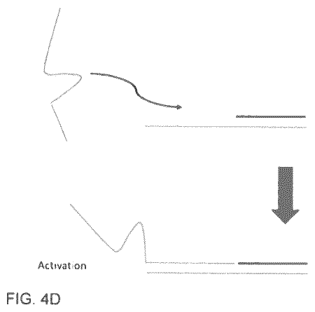

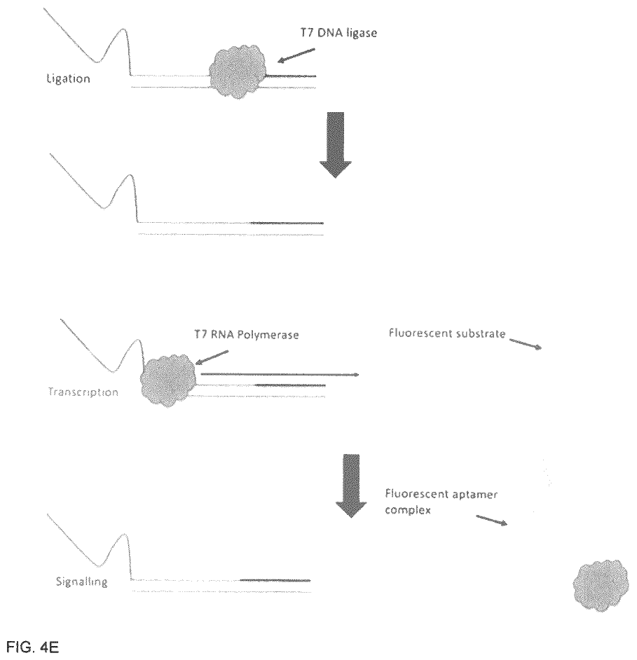

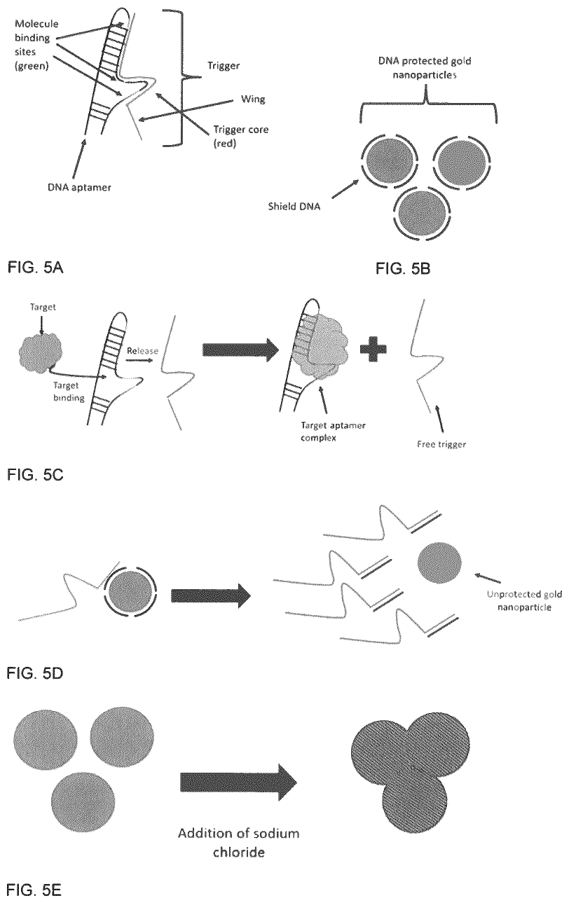

As discussed above, embodiments in which the trigger comprises a single wing are shown schematically in FIGS. 4 and 5.

In the embodiment shown in FIG. 4, the aptamer binds to the trigger sequence as shown in FIG. 1. However, the biosensor system includes additional components, specifically a fluorescent aptamer template, in this case, a template for transcription by the T7 promoter, a short partial primer corresponding to a first region of the T7 promoter (FIG. 4A), T7 DNA ligase, T7 RNA polymerase and a fluorescent substrate.

As can be seen in FIGS. 4B and 4C, once the trigger is dislodged or dislocated or released from the aptamer by the aptamer binding to its target, the wing or sensor binding region of the trigger binds to a second region of the T7 promoter. This is shown in the bottom panel of FIG. 4C, wherein the T7 promoter is bound at a second region thereof by the sensing binding sequence region of the trigger and a first region thereof is bound by the short partial primer. Furthermore, as shown in the bottom panel of FIG. 4C, there is a gap between these two binding regions which is sealed by the action of T7 ligase, shown in the top panel of FIG. 4D.

Following the action of T7 ligase, the annealed trigger and short partial primer act as a suitable promoter for T7 RNA polymerase transcription, which is capable of producing multiple RNA transcripts.

In the embodiment shown in FIG. 4D, the RNA transcript produced from the fluorescent aptamer template is an RNA molecule or strand that binds a fluorescent substrate. Furthermore, this binding or formation of a fluorescent substrate-RNA molecule complex results in the production of a detectable signal.

As will be apparent to one of skill in the art, the T7 transcription system allows for the amplification of a signal released by the introduction of a target molecule. The inactive state of the system involves a template for transcription consisting of an aptamer and trigger complex, a T7 promoter region, a fluorescent RNA aptamer, and an “assistant strand”. The assistant strand is complementary to a region of the T7 promoter so that the T7 promoter can be fully complemented upon the annealing of the trigger's wing, as discussed above. When a target molecule is present, the trigger is released from the aptamer. The trigger's wing sequence then anneals to the portion of the T7 promoter template not annealed by the assistant strand. Upon annealing, a T7 DNA ligase acts on it so that the wing and the assistant strands are ligated, completing the promoter complement. While this step has been shown to not be necessary, it significantly increases the efficiency. Specifically, in this embodiment, once the promoter sequence is completed, a T7 RNA polymerase transcribes the template strand, producing a fluorescent aptamer that binds to the fluorescent substrate, producing a fluorescent signal. It is of note that they are a number of such combinations known in the art and that these are within the scope of the invention.

In the embodiment shown in FIG. 5, the biosensor comprises the aptamer and trigger combination shown in FIG. 1 (FIG. 5A). However, in this embodiment, the sensing sequence binding region is illustrated as a single wing and the reporter is gold nanoparticles bound by a shield DNA (FIG. 5B). As discussed herein, in this example, on displacement of the trigger from the aptamer by the target (FIG. 5C), the sensing sequence binding region of the trigger binds to the shield DNA, forming a trigger:shield DNA duplex (FIG. 5D), which exposes the gold nanoparticle for detection (FIG. 5E).

Specifically, the gold nanoparticle system allows for colorimetric detection of a target molecule that can be visualized with the naked eye. This is possible due to the colorimetric properties of gold nanoparticles in which free nanoparticles are red but aggregated ones are blue. As discussed above and as shown in FIG. 5, the inactive state involves a trigger bound to an aptamer and gold nanoparticles with “shield” DNA adsorbed onto the surface. This shield DNA prevents the aggregation of the nanoparticles. When the target molecule binds to the aptamer, the trigger is released. Upon release, the wing of the trigger binds to the shield DNA, removing it from the nanoparticles, leaving the nanoparticles unprotected. Once unprotected, the addition of sodium chloride then leads to the aggregation of the nanoparticles, causing them to change colour. The degree at which it turns blue is proportional to the amount of target molecule present.

As can be seen from the data discussed in the examples, this system responds sensitively and specifically to its intended target. As discussed above, this sensitivity is enhanced by the use of the trigger, which acts as an intermediate or intermediary between the aptamer and the reporter. Specifically, in some embodiments, the trigger can be used in an amplification step which greatly enhances the sensitivity of the biosensor system.

As discussed herein, the design of the system, specifically, the use of the trigger as an intermediary, also greatly enhances the flexibility of the system, as a wide variety of reporters can be used, as discussed herein.

Standard curves performed for kanamycin and cocaine have shown a dose-dependent response to various concentrations of the target. Specificity assays have been used to demonstrate the ability of the system to produce signals only for their intended targets. Accordingly, these data, generated with different biosensors based on different aptamers, clearly demonstrate the robustness and wide applicability of the biosensors of the invention.

The invention will now be further explained and/or elucidated by way of examples; however, the invention is not necessarily limited to or by the examples.

### Example 1—Detection of Ampicillin

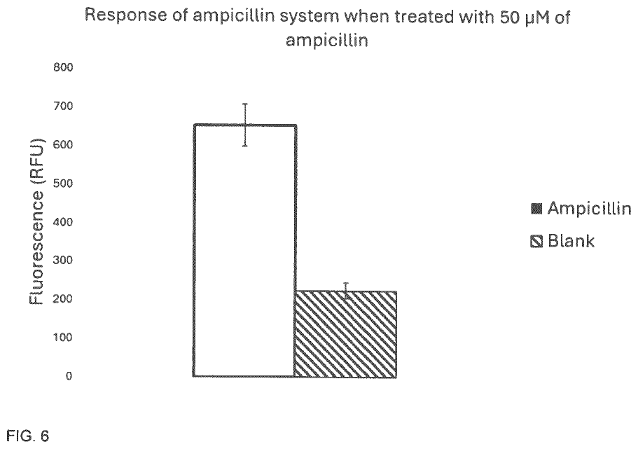

In this experiment, an ampicillin aptamer biosensor system was designed. The biosensor was exposed to 50 μM ampicillin and fluorescence was recorded as discussed above. The data is shown in Table 1 and the results shown graphically in FIG. 6. As can be seen, the ampicillin produced a significantly stronger signal than the blank or negative control.

### Example 2—Detection of Kanamycin

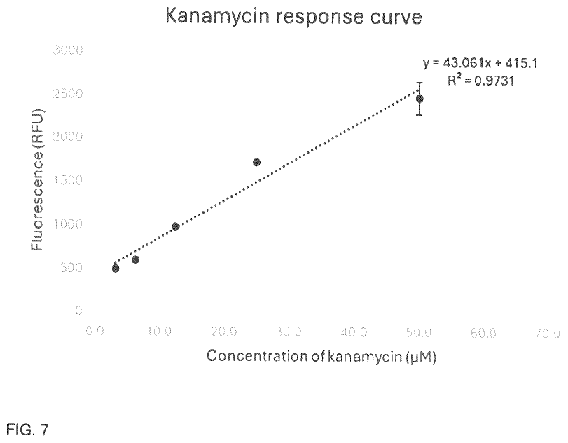

In this experiment, different doses of kanamycin (0-100 μM) were added to the biosensor system and fluorescence was determined as discussed above. The data is shown in Table 2 and the results are shown graphically in FIG. 7. As can be seen, the kanamycin dose response is also linear and proportional to the amount of kanamycin added to the biosensor system.

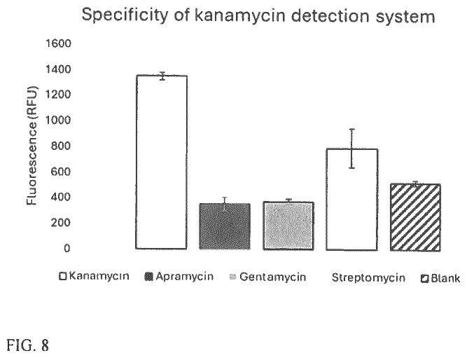

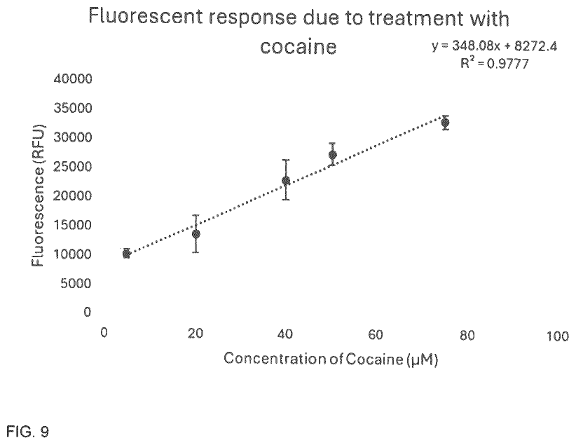

Shown in FIG. 8 is a second experiment with kanamycin wherein fluorescence produced from the kanamycin biosensor in the presence of kanamycin was compared to apramycin, gentamycin and streptomycin. As can be seen, both apramycin and gentamycin produced fluorescence signals that were below the blank/negative control while streptomycin produced a signal that was approximately half of the intensity of kanamycin (Table 3).

### Example 3—Detection of Cocaine

In this experiment, different doses of cocaine (0-150 μM) were added to the biosensor system and fluorescence was determined as discussed above. The data is shown in Table 4 and the results are shown graphically in FIG. 8. As can be seen, the cocaine dose response is also linear and proportional to the amount of cocaine added to the biosensor system.

While the preferred embodiments of the invention have been described above, it will be recognized and understood that various modifications may be made therein, and the appended claims are intended to cover all such modifications which may fall within the spirit and scope of the invention.

TABLE 1
Ampicillin data

| | Fluorescence | | | Average | SD | % CV | | | |
|---|---|---|---|---|---|---|---|---|---|
| Amp | 712 | 647 | 602 | 653.667 | 55.3022 | 8.46031 | 1.05481 | −0.1205 | −0.9343 |
| Blank | 248 | 218 | 208 | 224.667 | 20.8167 | 9.26558 | 1.1209 | −0.3203 | −0.8006 |

TABLE 2
Kanamycin data

| Concentration (uM) | Fluorescence | | | Average | SD |
|---|---|---|---|---|---|
| 100 | 2780 | 2895 | 2988 | 2887.7 | 104.19 |
| 50.0 | 2403 | 2311 | 2676 | 2463.3 | 189.83 |
| 25.0 | 1722 | 1723 | 1714 | 1719.7 | 4.9329 |
| 12.5 | 956 | 994 | 975 | 975 | 19 |
| 6.25 | 619 | 596 | 570 | 595 | 24.515 |
| 3.13 | 478 | 519 | 485 | 494 | 21.932 |
| 1.56 | 628 | 599 | 537 | 588 | 46.487 |
| 0.78 | 1245 | 645 | 806 | 898.67 | 310.55 |
| 0.39 | | 553 | 554 | 553.5 | 0.7071 |
| 0.20 | 256 | 263 | 219 | 246 | 23.643 |
| 0.10 | 263 | 357 | 215 | 278.33 | 72.231 |
| 0 | | 232 | 223 | 227.5 | 6.364 |

TABLE 3
Specificity of aptamer for kanamycin against
apramycin, gentamycin and streptomycin

| | fluorescence | | | | | |
|---|---|---|---|---|---|---|
| | replicate | replicate | replicate | | | |
| Sample | 1 | 2 | 3 | average | SD | % CV |
| kanamycin | 1371 | 1315 | 1364 | 1350 | 30.51229 | 2.26017 |
| apramycin | 401 | 294 | 362 | 352.3333 | 54.15102 | 15.36926 |
| gentamycin | 357 | 398 | 362 | 372.3333 | 22.36813 | 6.007556 |
| streptomycin | 768 | 953 | 657 | 792.6667 | 149.5337 | 18.86464 |
| blank | 540 | 521 | 498 | 519.6667 | 21.03172 | 4.047156 |

TABLE 4
Cocaine data

| Concentration (uM) | Fluorescence | | | Average | SD | |
|---|---|---|---|---|---|---|
| 150 | 34426 | 36793 | 35669 | 35629.3 | 1184 | top plateau |
| 100 | 31294 | 35019 | 33714 | 33342.3 | 1890.11 | |
| 75 | 30593 | 36973 | 31839 | 33135 | 3381.69 | |
| 50 | 25310 | 31248 | 25930 | 27496 | 3264.08 | |
| 40 | 22287 | 23019 | 23771 | 23025.7 | 742.022 | |
| 20 | 13595 | 13514 | 13818 | 13642.3 | 157.43 | |
| 5 | 10056 | 10318 | 10222 | 10198.7 | 132.549 | |
| 2.5 | 9670 | 10854 | 9883 | 10135.7 | 631.145 | |
| 1.25 | 10145 | 9824 | 10313 | 10094 | 248.457 | bottom plateau |
| 0.625 | | 9956 | 9930 | 9943 | 18.3848 | |
| 0 | 11842 | 9517 | 10284 | 10547.7 | 1184.71 | |

| | |
|---|---|
| LOD | 11.16483076 uM |
| LOD in mg/L | 3.386884906 uM |
| LLOQ in mg/L | 24.30348986 uM |
| LLOQ in mg/L | 7.37253656 uM |

1. A biosensor for detecting a compound of interest, said biosensor comprising:
an aptamer, said aptamer comprising a target binding region configured to be bound by or to bind to a compound of interest;
a sensor, said sensor comprising a sensing sequence region and a detectable reporter, said sensor having a reporting configuration wherein the reporter is detected and a non-reporting configuration; and
a trigger, said trigger comprising an aptamer binding region that binds to the target binding region and at least one sensing sequence binding region that binds to the sensing sequence, said trigger having an aptamer binding configuration wherein the aptamer binding region is bound to the target binding region of the aptamer and a sensor binding configuration wherein the at least one sensing sequence binding region is bound to the sensing sequence region of the sensor.

2. The biosensor according to claim 1 wherein the trigger is a single-stranded nucleic acid molecule.

3. The biosensor according to claim 2 wherein the single-stranded nucleic acid molecule is a single-stranded DNA molecule.

4. The biosensor according to claim 1 wherein the trigger comprises two sensing sequence binding regions, each sensing sequence binding region extending from one end of the aptamer binding region.

5. The biosensor according to claim 1 wherein the sensor is a single-stranded nucleic acid molecule.

6. The biosensor according to claim 5 wherein the sensor is a single-stranded DNA molecule.

7. The biosensor according to claim 1 wherein the sensing sequence region comprises two complementary sequence regions that are configured to form a stem structure separated by a non-complementary sequence region.

8. The biosensor according to claim 1 wherein:
the trigger comprises a 5′ sensing sequence binding region extending upstream from a 5′ end of the aptamer binding region and a 3′ sensing sequence region extending downstream from a 3′ end of the aptamer binding region,
the sensing sequence region comprises a 5′ sensor end complementary sequence region and a 3′ sensor end complementary sequence region that are configured to form a stem structure separated by a non-complementary sequence region, and
the 5′ sensing sequence binding region is complementary to the 3′ sensor end complementary sequence region and the 3′ sensing sequence binding region is complementary to the 5′ sensor end complementary sequence region such that the 5′ sensing sequence binding region can anneal to the 3′ sensor end complementary sequence region and the 3′ sensing sequence binding region can anneal to the 5′ sensor end complementary sequence region.

9. The biosensor according to claim 1 wherein the detectable reporter is a molecular beacon.

10. The biosensor according to claim 1 wherein the molecular beacon comprises a fluorophore bound to a first end of the sensor and a quencher bound to the second end of the sensor.

11. A method for detecting a compound of interest with a biosensor comprising:
providing a biosensor comprising:
an aptamer, said aptamer comprising a target binding region configured to be bound by or to bind to a compound of interest;
a sensor, said sensor comprising a sensing sequence region and a detectable reporter, said sensor having a reporting configuration wherein the reporter is detected and a non-reporting configuration; and
a trigger, said trigger comprising an aptamer binding region that binds to the target binding region and at least one sensing sequence binding region that binds to the sensing sequence, said trigger having an aptamer binding configuration wherein the aptamer binding region is bound to the target binding region of the aptamer and a sensor binding configuration wherein the at least one sensing sequence binding region is bound to the sensing sequence region of the sensor;
to a sample suspected of comprising the compound of interest, adding the biosensor wherein the biosensor is configured such that the sensor is in the non-reporting configuration and the trigger is in the aptamer binding configuration;
incubating the sample comprising the biosensor under conditions such that the compound of interest, if present, binds to the target binding region of the aptamer of a respective one biosensor,
said binding of the compound of interest to the target binding region of the aptamer of the respective one biosensor releasing the aptamer binding region of the trigger from binding to the target binding region of the aptamer;
said released trigger binding to the sensing sequence region of the sensor such that the at least one sensing sequence binding region of the trigger interacts with and binds to the sensing sequence region of the sensor,
said binding of the released trigger to the sensing sequence region converting the trigger to the sensor binding configuration and converting the sensor to the reporting configuration; and
detecting the reporter.

12. The method according to claim 11 wherein the trigger is a single-stranded nucleic acid molecule.

13. The method according to claim 12 wherein the single-stranded nucleic acid molecule is a single-stranded DNA molecule.

14. The method according to claim 11 wherein the trigger comprises two sensing sequence binding regions, each sensing sequence binding region extending from one end of the aptamer binding region.

15. The method according to claim 11 wherein the sensor is a single-stranded nucleic acid molecule.

16. The method according to claim 15 wherein the sensor is a single-stranded DNA molecule.

17. The method according to claim 11 wherein the sensing sequence region comprises two complementary sequence regions that are configured to form a stem structure separated by a non-complementary sequence region.

18. The method according to claim 11 wherein:
the trigger comprises a 5′ sensing sequence binding region extending upstream from a 5′ end of the aptamer binding region and a 3′ sensing sequence region extending downstream from a 3′ end of the aptamer binding region,
the sensing sequence region comprises a 5′ sensor end complementary sequence region and a 3′ sensor end complementary sequence region that are configured to form a stem structure separated by a non-complementary sequence region, and
the 5′ sensing sequence binding region is complementary to the 3′ sensor end complementary sequence region and the 3′ sensing sequence binding region is complementary to the 5′ sensor end complementary sequence region such that the 5′ sensing sequence binding region can anneal to the 3′ sensor end complementary sequence region and the 3′ sensing sequence binding region can anneal to the 5′ sensor end complementary sequence region.

19. The method according to claim 11 wherein the detectable reporter is a molecular beacon.

20. The method according to claim 11 wherein the molecular beacon comprises a fluorophore bound to a first end of the sensor and a quencher bound to the second end of the sensor.
